(19) United States
(12) Patent Application Publication
(10) Pub. No.: US 2017/0166503 A1
(43) Pub. Date: Jun. 15, 2017

(54) ECOLOGICAL AND ECONOMIC METHOD AND APPARATUS FOR PROVIDING HYDROGEN-BASED METHANOL

(71) Applicant: Swiss Liquid Future AG, Meggen (CH)

(72) Inventor: Peter Grauer, Meggen (CH)

(21) Appl. No.: 14/970,488

(22) Filed: Dec. 15, 2015

(51) Int. Cl. C07C 29/152; F02M 25/00; C10L 10/02; C10L 10/06; C07C 29/151; C10L 1/182

(52) U.S. Cl. CPC C07C 29/152; C07C 29/1518; C10L 1/1824; C10L 10/02; C10L 10/06; F02M 25/00; C10L 2290/42; C10L 2290/38; C10L 2270/026

(57) ABSTRACT

Method and device for providing and using a methanol-containing liquid.

### FIELD OF THE INVENTION

The present application relates to a method and an apparatus for providing a hydrogen-based methanol. Key aspects of the invention are ecological, economic and security considerations.

### BACKGROUND OF THE INVENTION

Some experts are propagating so-called “electric mobility” as the solution to overcome many of today's environmental problems caused by the emission of carbon dioxide, NOx and other harmful pollutions. There is one important issue, however, which is causing a serious delay of the success of electric vehicles.

In order to make the electric power available at many different locations, the electric power grid needs to be extended and upgraded. The required investment in the grid infrastructure is going to be huge. In some countries, there is a certain consumer resistance against the installation of additional pylons, power lines and transformers.

Partnerships were forged with communities, governments, and key industry actors in order to develop plans and in order to create the infrastructure needed. But there is still a long way to go.

This means that there is an immediate need for solutions which help to provide for a transition from today's petrol and diesel based economy to clean energy solutions. The electric mobility might eventually be one contribution towards a clean energy solution. The hydrogen mobility, which is propagated and supported by some experts, could be another clean energy solution.

It is believed that methanol, in particular synthetic methanol, is going to become an important if not essential element of the above-mentioned transition.

There are a number of patent applications and patents which relate to the production of methanol. See for example:
International Patent Application PCT/EP2010/064948 (see EP2624947), which was filed on 6 Oct. 2010 under the title “VERFAHREN UND ANLAGE ZUR SYNTHESE VON KOHLENWASSERSTOFF” (METHOD AND SYSTEM FOR SYNTHESIZING HYDROCARBON).
The European Patent Application EP11152947.5, which was filed on 1 Feb. 2011 under the title “VERFAHREN ZUR BEREITSTELLUNG UND ZUM EINSETZEN EINES ALKOHOLS UND VERWENDUNG DES ALKOHOLS ZUR LEISTUNSSTEIGERUNG EINER VERBRENNUNGSKRAFTMASCHINE” (METHOD FOR PROVIDING AND USING AN ALCOHOL AND USE OF THE ALCOHOL TO INCREASE THE PERFORMANCE OF AN INTERNAL COMBUSTION ENGINE),
The European Patent Application EP11155310.3 (see EP2438982), which was filed on 22 Feb. 2011 under the title “VERFAHREN ZUR BEREITSTELLUNG UND ZUM EINSETZEN EINES ALKOHOLS UND VERWENDUNG DES ALKOHOLS ZUR WIRKUNGSGRAD- UND LEISTUNGSSTEIGERUNG EINER VERBRENNUNGSKRAFTMASCHINE” (METHOD FOR PROVIDING AND USING AN ALCOHOL AND USE OF THE ALCOHOL TO INCREASE THE EFFICIENCY AND PERFORMANCE OF AN INTERNAL COMBUSTION ENGINE), and
The European Patent Application EP11167622.7, which was filed on 26 May 2011 under the title “VERFAHREN UND VORRICHTUNG ZUR BEREITSTELLUNG UND ZUM EINSETZEN VON WASSERSTOFF-BASIERTEM METHANOL ZU DENITRIFIZIERUNGSZWECKEN” (METHOD AND DEVICE FOR PROVIDING AND USING HYDROGEN-BASED METHANOL FOR DENITRIFICATION PURPOSES).

Carbon dioxide CO₂ is a chemical compound comprising carbon and oxygen. Since the beginning of industrialization, the CO₂ component in the atmosphere has significantly risen. The main causes of this are the CO₂ emissions caused by humans—so-called anthropogenic CO₂ emissions. The carbon dioxide in the atmosphere absorbs a part of the thermal radiation. This property makes carbon dioxide a so-called greenhouse gas (GHG) and one of the contributing causes of the global greenhouse effect.

For these and other reasons, research and development is currently being performed in greatly varying directions in order to find a way to reduce the anthropogenic CO₂ emissions. In particular in connection with power generation, which is frequently performed by the combustion of fossil fuels, such as coal, oil, or gas, but also with other combustion processes. For example, in garbage burning, there is a great demand for reduction of the CO₂ emission. Currently, approximately 30 billion tons of CO₂ are discharged into the atmosphere per year by such processes.

It is considered to be a problem that CO₂ arises during the combustion of fossil fuels. In addition, the fossil resources, which are finite, are irrevocably consumed. Therefore, research is being performed in greatly varying directions in order to reduce the consumption of vehicles or to develop vehicles which are driven completely using regenerative power.

Methanol is a particularly advantageous substrate, since it is fully soluble in water and is easily biologically degradable. However, methanol has been produced up to this point from fossil raw materials, for example, from natural gas in most cases. Numerous methods and reactors for producing methanol are known. Corresponding exemplary patent applications and patents are listed hereafter:
EP 0 790 226 B1;
WO 2010/037441 A1;
EP 4 483 919 A2.

### SUMMARY OF THE INVENTION

For the applications mentioned at the beginning, the demand exists for the provision of methanol which is CO₂-neutral and cost-effective to produce. In addition, the methanol production is not to compete with food production and is not to require agricultural land, as in the production from biomass.

The object presents itself of developing a corresponding method and a corresponding apparatus for providing methanol especially for the purpose to provide ecologically and economically mobility solutions.

Therefore, a novel method is proposed according to the invention, which relates to the production of synthetic methanol and preferably with the consumption of synthetic methanol in vehicles.

The method comprises the following steps:
providing a carbon-containing gas, preferably a carbon dioxide gas, as a carbon supplier,
providing a hydrogen gas by means of an electrolyzer,
providing a gaseous starting material, which comprises the carbon dioxide gas and hydrogen gas,
introducing the starting material into a catalytic reactor in order to synthesize a methanol containing liquid in a catalytic-synthetic way,
providing the methanol-containing liquid at an output-side of the reactor, said methanol-containing liquid being a mixture of methanol and water,
   wherein said electrolyzer and said catalytic reactor are part of an on-site facility of a water power plant and wherein said power plant is electrically connectable to said electrolyzer and/or to said catalytic reactor so as to supply electric energy consumed by the electrolyzer and/or the catalytic reactor.

The method and apparatus presented herein will become an essential part of a smarter and more sustainable future. The method and apparatus presented herein offer multiple environmental, economic, and energy system benefits.

The invention presented herein is considered to be a transformative technology and enables a business model which will help in the transition towards an electric mobility and possibly also a hydrogen technology.

The invention offers an innovative solution which is going to create new opportunities for electric mobility and for other forms of mobility in the future.

The goal of the invention is also to provide a system which functions autonomously, i.e., independently of the power grid. In particular, this relates to a method in which at least the power requirement, which exists in order to provide the hydrogen component (H₂) electrolytically, is generated locally in a water power plant (called hydroelectric power plant) or in its immediate surroundings. In preferred embodiments, the power required for other components/facilities (e.g. the reactor) of the on-site facility is provided by the hydroelectric power plant, too.

It is an economic advantage of the inventive solution that no fees or other charges need to be paid for using the regular power grid. These fees or charges are typically charged for the transmission of electric energy e.g. from a remote power plant to a local consumer.

It is an advantage of the invention that is can be realized as a stand-alone plant which can be located directly near by the sources of renewable electrical energy, namely a water power plant.

Preferred embodiments of the inventive on-site plant are designed so that they work independently from any other industrial infrastructure, such as the power grid.

In accordance with preferred embodiments of the invention, the carbon-containing gas is delivered to the on-site plant in cryogenic and pure condition. Preferably, CO₂ from biogenic sources is used as carbon-containing gas in connection with all embodiments of the invention.

The power is preferably generated in a steady stream water power plant because such a plant is designed to steadily provide electric output power.

Preferred embodiments of the invention use decentralized units for the production of methanol. In a particularly preferred embodiment of the invention, the water power plant and the methanol production facility are specifically designed for the purpose of producing synthetic methanol from electrically produced hydrogen.

The methanol, which is produced on-site of a water power plant, is advantageously being used a neutral synthetic fuel (e.g. in an engine which is specifically designed or adapted for the combustion of methanol) or it can be used for blending with another fuel (e.g. petrol) or it can be used for mixing with diesel and/or injection into a diesel engine.

The methanol, however, could also be used as fuel for fuel cells.

Preferably, the on-site plant of the invention is designed for continuous operation. It shuts down or reduces its power consumption only to perform maintenance or repair or, if needed, because of a reduced availability of water resources.

It is another advantage of the present invention that it is independent of the electric power grid. This could be an advantage for instance if the power grid is disturbed or down because of a cyber-attack or if the infrastructure of a power grid has been destroyed during a severe storm, or if it is damaged by a geo-magnetic storm from the sun or by a direct physical attack.

It is a further advantage of the invention that a water power plant can be erected at remote locations where no connection to the national power grid is feasible. The respective power plant together with a methanol production facility, in accordance with the present invention, in this case would form a independent solution which would function even if other elements of the national or transnational infrastructure for some reason is not available.

The electric power which is required for electric mobility cannot be handled by the existing power grid infrastructure. The present invention helps to establish and independent infrastructure and a related business model which will become an essential element of the future mobility. There are too many solutions which are depending on the existing infrastructure and which will be as vulnerable as this infrastructure is.

The invention is thus going to improve a region's or country's independence and it guarantees mobility even if other infrastructure is harmed or destroyed.

There is also the advantage that the production, distribution and use of synthetic methanol is not in competition with the production of grain or other comestible goods, as is the case with bio-ethanol and other fuels produced from the crops in fast growing woods or other economic plants, for instance.

Another advantage is the fact that, due to the local production of synthetic methanol, the dependence on oil-producing countries is going to be reduced.

Alternatively, the hydrogen component (H₂) can to some extent be generated directly from methane-containing gas, for example. In this case, the electrolyser can have a smaller dimension/capacity and the electric power consumption is going to be reduced. In other words, a combination of an electrolysis plant and hydrogen provision from locally existing gas is also possible.

The invention is intentionally based on carbon dioxide and hydrogen as the starting materials, since the carbon dioxide is “recycled” in this way. This helps to improve the carbon-footprint.

The carbon dioxide can be obtained from exhaust gases or generated from biomass, so that the methanol, which is synthesized from these starting materials catalytically, can be considered to be CO₂-neutral.

According to the invention, synthesis gas which comprises H₂ and CO₂ is converted efficiently and in an economically advisable manner into methanol.

According to the invention, the carbon dioxide is caused to react with the hydrogen component in the presence of a catalyst, in order to convert it into a methanol-water mixture. The respective process step is thus referred to as synthesizing a methanol containing liquid in a catalytic-synthetic way.

The carbon dioxide is preferably taken from a combustion process or an oxidation process of carbon or hydrocarbons by means of CO₂ separation. For example, CO₂ can originate from a local fabrication plant. If the CO₂ originates from a local fabrication plant, the plant according to the invention is fully autonomous, since neither energy nor other materials must be externally delivered or supplied depending on the design of the plant and depending on the environmental conditions.

The method according to the invention for providing the methanol-containing liquid is controlled by an appropriate process control and the individual processes are technically “linked” to one another so that
the total yield and the quality of the methanol are ideal for the intended purpose,
and/or the (total) CO₂ emission is as minimal as possible,
and/or the most consistent and long-term possible plant workload is achieved, taking into account the capacity of the water power plant,
and/or the product-specific investment and operating costs are as minimal as possible.

Locally provided power is essential for the invention to give an competitive edge.

Using a corresponding plant, a methanol-water mixture is preferably produced as a liquid which can be stored and transported. The locally provided electric power and/or renewable power is chemically converted into a liquid which is relatively simple to store and transport. This liquid (called methanol-containing liquid) is preferably used for driving vehicles.

The production of the methanol-containing liquid as a mixture which can be stored and transported relatively simply can be phased down or even interrupted at any time. The processing plant parts for producing the methanol-containing liquid can be phased down or shut down relatively easily and rapidly. The ultimate decision is in the scope of responsibility of the operator of the plant.

All embodiments of the invention are based on the hydrogen generation with the aid of electrical energy, which is locally (i.e., on site in the area of a water power plant) generated. Hydrogen which is generated on site via electrolysis and/or from waste materials, for example, does not need to be stored or highly compressed or cryogenically liquefied and transported over long distances, but rather serves as an intermediate product, which is preferably supplied at the location of its generation immediately or soon to the above-mentioned reaction to generate methanol.

A novel method relevant to power engineering is provided according to the invention in consideration of corresponding power engineering, industrial, and economic parameters, together with the requirement for careful use of all material, energetic, and economic resources.

Further advantageous embodiments can be inferred from the description, the figures, and the dependent claims.

### BRIEF DESCRIPTION OF THE DRAWINGS

Various aspects of the invention are schematically shown in the drawings.

FIG. 1A shows a schematic diagram which summarizes some of the basic steps of the invention;

FIG. 1B shows a schematic diagram which shows details of a water power plant to be used in connection with the invention;

FIG. 1C shows a schematic diagram which summarizes further steps of the invention;

FIG. 2 shows a schematic diagram of two different scenarios for the use of the methanol-containing liquid of the invention; and

FIG. 3 shows a schematic view of a vehicle which is equipped so as to be able to use methanol-containing liquid of the invention.

### DETAILED DESCRIPTION OF THE INVENTION

The invention relates to methanol-water mixtures (MeOH1) and to pure methanol MeOH. The methanol-water mixture MeOH1 is also referred to as methanol-containing liquid.

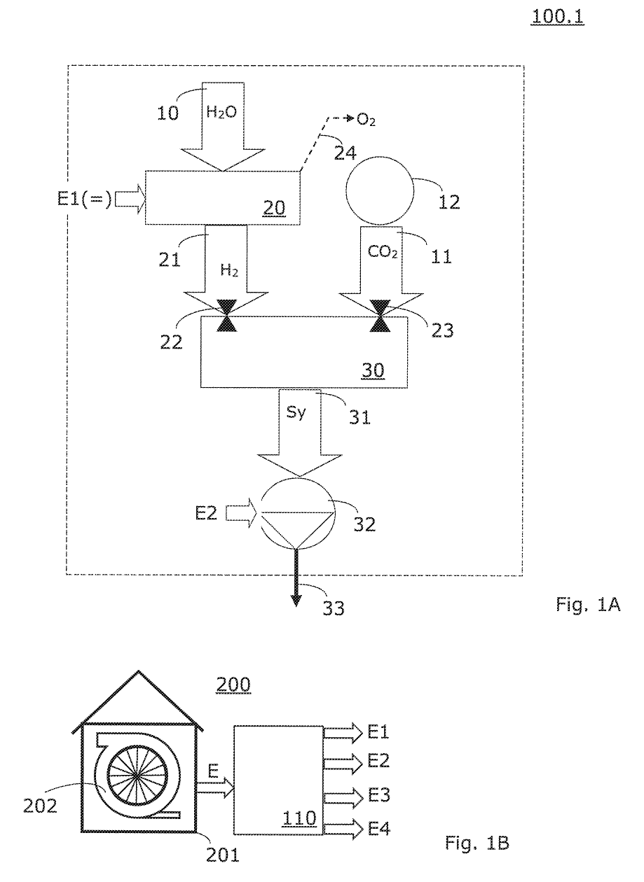

The term mixture MeOH1 is used here since the product which is provided at the output side 42 of a reactor 40 (see FIG. 1C) does not consist of 100% methanol. Rather, it is a so-called physical mixture of methanol and water.

A preferred embodiment of the invention is depicted in FIGS. 1A-1C. These figures are a combination of the details of a flow-chart and the details of a block diagram. That is, these figures combine structural elements with functional elements in order to be able to explain the essential aspects of the invention.

The method of the invention is specifically designed for providing a methanol containing liquid MeOH1. Details are illustrated in FIGS. 1A, 1B, and 1C. The method comprises at least the following steps, which are not necessarily carried out in the below sequence:
providing a carbon-containing gas 11, preferably a carbon dioxide gas CO₂, as a carbon supplier,
providing a hydrogen gas 21 H₂ by means of an electrolyzer 20,
providing a gaseous starting material Sy33, which comprises the carbon dioxide gas 11 and hydrogen gas 21,
introducing the starting material Sy into a catalytic reactor 40 (see FIG. 1C) in order to synthesize a methanol containing liquid MeOH1 in a catalytic-synthetic way,
providing the methanol-containing liquid MeOH1 at an output-side 42 of the reactor 40, said methanol-containing liquid MeOH1 being a mixture of methanol MeOH and water H₂O.

The electrolyzer 20 and the catalytic reactor 40 are part of an on-site facility 100.1 (see FIG. 1A), 100.2 (see FIG. 1C) of a water power plant 200 (see FIG. 1B). The power plant 200 is electrically connectable to the electrolyzer 20 and/or to the catalytic reactor 40 so as to supply electric energy E1 consumed by the electrolyzer 20 and/or to supply electric energy E3 consumed by the catalytic reactor 40.

FIG. 1B is a simplified sketch of a water power plant 200. A power house 201 is depicted with an electric generator 202. On the right hand side a substation 110 is shown. Electric energy provided by the generator 202 is fed to the substation 110, as indicated by an arrow E. The substation 203 might comprise transformers, power breakers and for instance a rectifier. In the sketch of FIG. 1B the substation 110 has four power ports or terminals E1, E2, E3 and E4. The sketch of FIG. 1B is just an example by means of which some of the essential aspects of the invention are highlighted.

The on-site plant, which includes the facilities 100.1 and 100.2, comprises a number of energy consumers. The most important consumers are described hereinafter. There is a number of other elements which are powered by electric energy and which are necessary for the plant 100.1, 100.2 to operate reliably. Examples are pumps, sensors, heating elements, computers, electrical cabinets and so forth. In a preferred embodiment of the invention all these elements are powered by the water power plant 200.

The electrolyzer 20 of plant 100.1, 100.2 has the highest consumption of electric power since it splits water into hydrogen gas H₂ and oxygen gas O₂. The electrolyzer 20 is by far the largest consumer of electric energy. Preferably, an electrolyzer 20 is used which is operated with direct current. The substation 110 thus comprises at least one rectifier which is suitable to provide the right electric power E1 to the terminals of the electrolyzer 20. In FIG. 1A, an arrow E1=is pointing towards the electrolyzer 20. This arrow E1=symbolizes the electric power supply of the electrolyzer 20 with direct current.

The second largest consumer is the reactor 40 with its peripheral elements, such as heater 43, pumps, valves, sensors and so forth. The chemical reaction takes place while starting material Sy (preferably a syngas) travels through the catalytic reactor 40. The catalyzer inside the reactor 40 boosts/supports the chemical reaction by means of which the methanol containing liquid MeOH1 is synthesized in a catalytic-synthetic way. The reaction as such as an exothermal reaction which does not consume external energy. However, for this reaction to be initiated and maintained, certain conditions are to be ensured. There needs to be a well-defined temperature inside the reactor 40 and the pressure is to be within a certain range. When initiating the chemical reaction, energy might have to be provided to trigger the process. Once the process is running, excess energy in the form of heat is provided which needs to be removed in order to ensure a stable operation of the reactor 40. As indicated in FIG. 1C, the reactor 40 comprises a cooling system where water (or some other fluid) is fed into the space which is enclosing the reactor pipes or the reactor chamber of the reactor 40. The reference number 46 is assigned to a pipe which is employed in order to ensure that the water circulates through the reactor 40. A water heater 44 might be necessary to increase the temperature of the water and the reactor 40. This heater 44, if installed, might be powered by electric energy E3.

In addition, a compressor 32 (see FIG. 1A) might be employed at the input side 41 of the reactor 40 in order to compress the gas Sy. The compressor 32 is preferably also fed by the water power plant 200, as indicated by the arrow E2 in FIGS. 1A and 1B. At the output side 42 of the reactor 40 there is a distiller 50 which is employed in order to produce pure methanol MeOH from the methanol containing liquid MeOH1. The distiller 50 consumes electric energy since it is designed to heat up the MeOH1. The distiller 50 is preferably also powered by the water power plant 200, as indicated by the arrow E4 in FIGS. 1B and 1C.

The plant 100.1, 100.2 of all embodiments of the invention is either designed in order to provide just the methanol-containing liquid MeOH1 at the output-side 42 of the reactor 20, or it is designed to provide pure methanol MeOH at the output side 63 of a distiller 50. If needed, the plant 100.1, 100.2 might also be designed so as to provide the methanol-containing liquid MeOH1 as well as pure methanol MeOH.

The electrolyzer 20 and/or the catalytic reactor 40 are operated in an off-line mode, preferably in a fully autonomous off-line mode. A fully autonomous mode is a mode where no electric energy is supplied from outside the plant. In this case the plant 100.1, 100.2 together with the plant 200 form an isolated application.

In a less preferred embodiment, some auxiliary energy might be provided from the outside so as to be able to run for instance the computers, networks, monitoring systems and the like if the water power plant 200 should be in a maintenance mode.

The larger-scale power consumers of the plant 100.1, 100.2 should not be supplied by electric energy from the outside.

That is, at least the electrolyzer 20 and/or the catalytic reactor 40 are operated in an off-line mode, preferably in a fully autonomous off-line mode. The expression off-line refers to the fact that these systems 20 and/40 are totally independent from the power grid or (high-)voltage grid.

Preferably, the carbon dioxide gas CO₂, which serves as carbon-containing gas, and the hydrogen gas H₂ are mixed so as to form a synthesis gas Sy, as illustrated in FIG. 1A. There are two valves 22, 23 or similar elements which can be controlled by a computer or another control unit. These valves 22 and 23 can be used to define the two gas flows so that the synthesis gas Sy at the output side 31 of the respective mixer 30 (see FIG. 1A) has the required gas ratios. There might be a compressor 32 situated downstream of the mixer 30, as illustrated in FIG. 1A.

These steps are being carried out on-site and are preferably electrically powered by the power plant 200. Preferably the carbon dioxide gas CO₂ and the hydrogen gas H₂ are combined with respective ratios so that the synthesis gas Sy at the output side 31 of the respective mixer 30 comprises 1 mole of carbon dioxide gas per 1 mole of the hydrogen gas.

Preferably, there is a pre-heater 43 employed at the input side 41 of the reactor 40, as illustrated in FIG. 1C. This pre-heater 43 might be electrically powered by the power plant 200 and/or it might be powered by (waste) heat of some other process of the plant 100.1, 100.2.

The reactor 40 might be cooled and/or heated by a circulating fluid (preferably water). The respective fluid connection carries the reference number 46 in FIG. 1C.

Preferably, there is a heater 44 included in the fluid system, as indicated in FIG. 1C. This heater 44 might be electrically powered by the power plant 200 and/or it might be powered by (waste) heat of some other process of the plant 100.1, 100.2. In FIG. 1C, the heater 44 is an electrically powered heater, as indicated by the arrow E3.

In a preferred embodiment, there is a buffer tank 60 provided at the output side 42 of the reactor 40. This tank 60 can for instance be used to (temporarily) store the methanol-containing liquid MeOH1. The tank 60, if present, is preferably situated between the reactor 40 and a distiller 50, as shown in FIG. 1C.

The distiller 50 is employed in order to increase the concentration of the methanol in the methanol-containing liquid MeOH1. Out an output 63 of the distiller 50 pure methanol MeOH is provided.

In a preferred embodiment, there is a buffer tank 61 provided at the output side 42 of the distiller 50. This tank 61 can for instance be used to (temporarily) store the methanol MeOH. In the present example, the methanol MeOH is made available at an output 62 (see FIG. 1C).

In preferred embodiments, this distiller 50 is electrically powered by the power plant 200. It might also be powered by (waste) heat of some other process of the plant 100.1, 100.2. In FIG. 1C, the distiller 50 is an electrically powered distiller, as indicated by the arrow E4.

In a preferred embodiment, the oxygen gas O₂, which is produced by the electrolyzer 20, is introduced into the water reservoir or stream of the power plant 200 so as to improve the water quality and/or the oxygen gas O₂ is introduced into the water downstream of the power plant 200 to increase the oxygen content for a healthier flora and fauna. In FIG. 1A there is an outlet port 24 which could be used to introduce the oxygen gas O₂ into water. This feature is optional.

The following constellation is particularly favorable because it combines the advantages of the methanol production plant 100.1, 100.2 driven/supplied by a power plant 200 with a new and independent distribution approach for the methanol MeOH1 and/or MeOH.

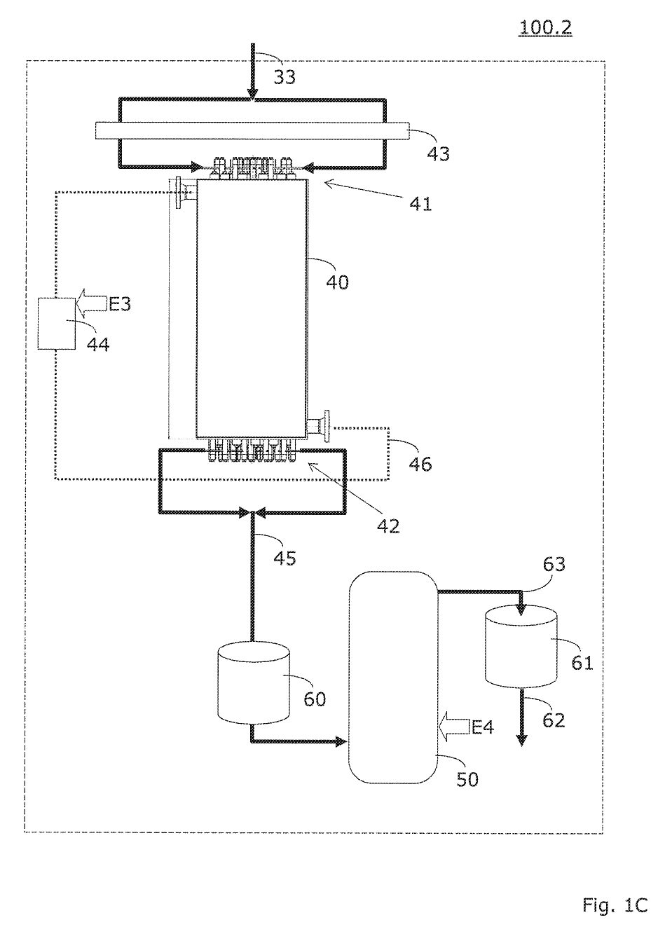

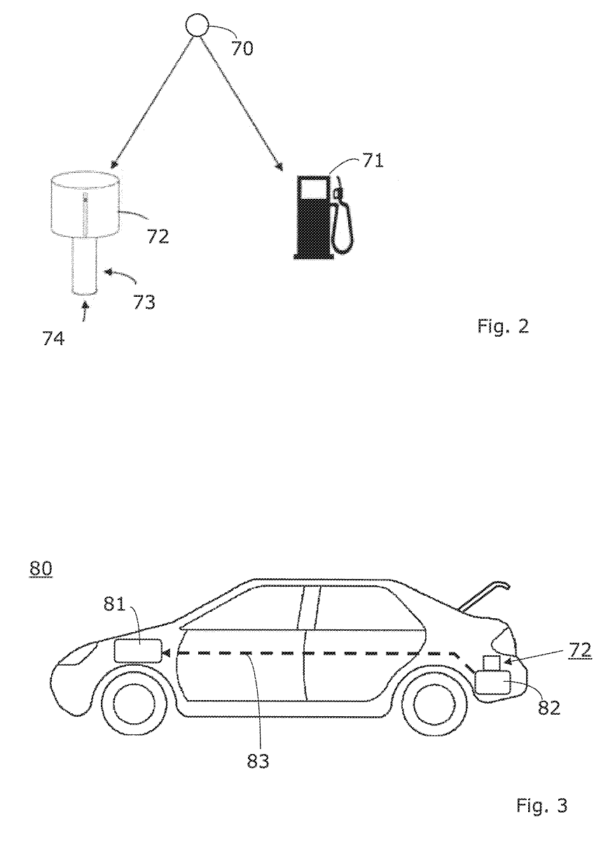

A solution takes advantage of the invention where the MeOH1 and/or MeOH is filled into small containers 72 for use inside a vehicle 80 (see FIGS. 2 and 3, for example). The respective container 72 should have a size and weight so that it still is portable for a human being (its weight should be less than 30 kg).

Preferred embodiments of the container 72 are designed so that the respective container 72 can be plugged into a vehicle 80 so that MeOH1 and/or MeOH is made available inside the vehicle 80 if needed.

The vehicle 80 could be an electrically powered car which carries batteries and an electric engine. This vehicle 80 in addition comprises a small combustion engine which is specifically designed so as to by fueled by MeOH1 and/or MeOH from the container 72. The combustion engine is mechanically connected to an electric generator in order to produce electric power if needed inside the vehicle 80. This setup is similar to what is known as range extender.

It is an advantage that for most purposes the vehicle 80 can be used in the electric mode where power is taken from the battery pack. If the vehicle 80 is used for a longer distance or for a longer period of time, a sufficient number of containers 72 are taken onboard so as to obtain the required independence as far as the range is concerned.

The containers 72 may also be used in cases where the MeOH1 and/or MeOH is used inside a vehicle 80 for cooling the intake air of a combustion engine or for cooling the compressed air of an (electrically powered) compressor of a combustion engine.

Instead of the distribution by means of containers 72 (see left hand side of FIG. 2), the MeOH1 and/or MeOH could also be made available at petrol stations (here illustrated by means of a petrol pump 71) for vehicles 80 which are designed for the combustion of methanol. Or diesel or petrol could be blended with MeOH1 and/or MeOH (in accordance with local rules and regulations) so as to be used as an alternative diesel or petrol which is cleaner than fossil diesel or fossil fuel.

The vehicle 80 might comprise a bay or port for receiving a nozzle 73 of the container 72. If the container 72 is inserted into the bay or port, it might be connected to a tank 82, as depicted in FIG. 3. The illustration in FIG. 3 is to be understood as an example only.

The tank 82 might have a fluid connection 83 (e.g. a fuel pipe with pump) to the combustion engine 81, as depicted in FIG. 3. The methanol, as taken from the container 72 could be used in the vehicle 80 of FIG. 3 for cooling the intake air of the combustion engine 81 or for cooling the compressed air of an (electrically powered) compressor of the combustion engine 81.

Details of a respective container and of a vehicle using such a container are given in the published patent application EP2758317.

In addition to ecological advantages, sustained cost advantages also result through the invention.

### REFERENCE NUMBERS

| | |
|---|---|
| Water (H₂O) supply | 10 |
| Carbon-containing gas (e.g. CO₂) supply | 11 |
| gas buffer tank | 12 |
| electrolyzer | 20 |
| hydrogen gas outlet | 21 |
| Flow control valve | 22 |
| Flow control valve | 23 |
| outlet port | 24 |
| Mixer/blender | 30 |
| Synthetic gas (Sy) supply | 31 |
| compressor | 32 |
| High-pressure feed port | 33 |
| Reactor | 40 |
| Input side | 41 |
| Output side | 42 |
| Pre-heater (optional) | 43 |
| Water heater (optional) | 44 |
| Methanol containing liquid (MeOH1) outlet | 45 |
| Distillation plant | 50 |
| Methanol tank I. (raw methanol containing liquid) | 60 |
| Methanol tank II. (pure methanol) | 61 |
| (MeOH) Output | 62 |
| Distribution point | 70 |
| Fuel dispenser | 71 |
| Container | 72 |
| Outlet, nozzle | 73 |
| Aperture | 74 |
| Vehicle | 80 |
| Engine | 81 |
| (buffer) tank | 82 |
| Fuel pipe | 83 |
| On-site plant (part 1) | 100.1 |
| On-site plant (part 2) | 100.2 |
| Water power plant | 200 |
| power house | 201 |
| Generator | 202 |
| Electric power | E |
| Substation | 110 |
| first electric power supply | E1 |
| second electric power supply | E2 |
| third electric power supply | E3 |
| fourth electric power supply | E4 |
| Methanol containing liquid | MeOH1 |
| Methanol | MeOH |

What is claimed is:

1. A method for providing liquid methanol comprising the following steps:
providing a carbon-containing gas as a carbon supplier,
providing a hydrogen gas by means of an electrolyzer,
mixing said carbon-containing gas and hydrogen gas to form a gaseous starting material (Sy),
introducing said starting material (Sy) into a catalytic reactor in order to synthesize a methanol containing liquid in a catalytic-synthetic way,
providing the methanol-containing liquid at an output-side of the reactor, said methanol-containing liquid being a mixture of methanol and water,
wherein said electrolyzer and said catalytic reactor are part of an on-site facility of a water power plant and wherein said power plant is electrically connectable to said electrolyzer and/or to said catalytic reactor so as to supply electric energy consumed by the electrolyzer and/or the catalytic reactor.

2. The method according to claim 1, wherein said electrolyzer and/or said catalytic reactor is/are operated autonomously without being connected to a power grid.

3. The method according to claim 1, wherein both said electrolyzer and/or said catalytic reactor are operated in an off-line mode.

4. The method according to claim 1, wherein said electrolyzer and/or said catalytic reactor are being constantly supplied with electric power generated by said water power plant.

5. The method according to claim 1, wherein said starting material (Sy) is compressed before being introduced into said catalytic reactor.

6. The method according to claim 1, wherein said methanol containing liquid is distilled to remove water.

7. The method according to claim 1, wherein a purifying process for a purification of the carbon-containing gas is carried out off-site before said carbon-containing gas is mixed with said hydrogen gas.

8. The method according to claim 7, wherein purified carbon-containing gas is delivered as cryogenic gas to an on-site gas buffer tank before being mixed with said hydrogen gas.

9. The method according to claim 1, wherein the methanol-containing liquid is delivered to at least one distribution point, plant or sales location so as to make it available for the use in vehicles.

10. The method according to claim 6, wherein the liquid methanol is:
used for the injection into a diesel engine of a vehicle, or
used for blending with petrol, said blending being carried out in a stationary plant or inside a vehicle, or
used in a combustion engine being designed in order to combust methanol.

11. The method according to claim 6, wherein the liquid methanol is filled into portable containers.

12. The method according to claim 11, wherein the portable containers are designed for being inserted or plugged into a vehicle and wherein the vehicle is enabled to take liquid from a container after it was inserted or plugged in.

13. The method according to claim 12, wherein the liquid methanol is used inside a vehicle for injection or blending with charge air or intake air supplied to a diesel engine, so as to reduce the nitrogen oxide emissions of the diesel engine.

14. The method according to claim 12, wherein the liquid methanol is used inside a vehicle for injection into the cylinders of the vehicle's diesel engine.

15. The method according to claim 12, wherein the liquid methanol is blended with diesel at an upstream point of the vehicle's injection pump.

16. The method according to one of the claim 14, wherein due to the evaporation of methanol inside the diesel engine the soot emission of the diesel engine is reduced.
